(19) United States
(12) Patent Application Publication
(10) Pub. No.: US 2007/0092483 A1
(43) Pub. Date: Apr. 26, 2007

(54) Surgical adhesive compostion and process for enhanced tissue closure and healing

(75) Inventor: Jacob Freas Pollock, Kensington, CA (US)

Correspondence Address: DERGOSITS & NOAH LLP, FOUR EMBARCADERO CENTER, SUITE 1450, SAN FRANCISCO, CA, 94111, US

(73) Assignee: Pollock Polymer Group

(21) Appl. No.: 11/584,140

(22) Filed: Oct. 20, 2006

Related U.S. Application Data
(60) Provisional application No. 60/729,133, filed on Oct. 21, 2005.

(51) Int. Cl. A61K 31/785; C08G 63/91

(52) U.S. Cl. 424/78.27; 525/54.1; 977/906

(57) ABSTRACT

A surgical tissue adhesive composition contains at least one 1,1-disubstituted electron-deficient olefin macromer. The adhesive composition of the invention has improved biocompatibility as well as controlled biodegradation characteristics and bioactivity. Adhesive co-monomer compositions contain at least one macromer with a pendant oligomer, polymer, or peptide chain as an acrylic ester of the reactive olefin. The polymers formed therefrom have a grafted brush-like nature. The composition is particularly useful for creating an adhesive bond at the junction of living tissue in surgical applications. The adhesive composition may further comprise co-monomer, co-macromer, cross-linker, or inter-penetrating polymer compounds containing peptide sequences that are bioactive or enzyme responsive. The peptide sequences are selected to promote tissue infiltration and healing in a particular biological tissue. The sequences may contain specific cell-adhesion, cell-signaling, and enzyme-cleavable domains. Furthermore, a degradable filler material may be included in the composition to create a reinforced composite. The filler preferably has a higher degradation rate than the polymer matrix, generating porosity upon degradation. The adhesive may further contain entrapped or incorporated drugs or biologics, including antibiotics or growth factors. The adhesive can be used to bind together the edges of living tissues during surgical procedures. The cured composition provides interfacial bonding and mechanical fixation while promoting tissue infiltration and replacement of the adhesive polymer.

This nonprovisional utility patent application claims priority under 35 U.S.C. § 119(e)(1) to provisional patent application No. 60/729,133, filed on Oct. 21, 2005.

### TECHNICAL FIELD

The present invention relates generally to compositions useful for adhering biological tissues. More specifically, the invention relates to adhesives for use in repairing tissue during medical surgical procedures. The invention additionally relates to methods of use of such compositions in surgical operating procedures.

### BACKGROUND OF THE INVENTION

Surgical adhesives are appealing alternatives and supplements to traditional methods of tissue closure such as sutures, staples, or other hardware. These materials can be used to join living tissue in order to promote and enhance the natural healing process. Such adhesives have the potential to decrease operating time, infection rates, inflammation, and foreign body response compared to traditional methods of closure. The use of effective tissue adhesives in surgical operations would lead to improved healing and rehabilitation with less scarring. Surgical adhesives have found use in various forms in such medical fields as orthodontics, craniofacial orthopedics, ophthalmology, cardiology, and cosmetic and reconstructive surgery. Surgical adhesives with enhanced performance would expand their use and allow for the development of new and improved medical procedures and treatments. These advances would serve to improve overall patient care and potentially lower health care costs.

Numerous materials have been used as tissue adhesives in research and clinical practice with varying degrees of success. Discovery and development of successful surgical adhesives have been limited due to challenges inherent to chemically reactive, biocompatible and bioactive materials for regenerative medicine. Current surgical adhesives suffer from either poor mechanical or biological integration with native tissue. Biologically derived adhesives, such as fibrin glues, promote healing but suffer from low interfacial bonding and internal strengths, limited sources, and potential antigenicity. On the other hand, use of stronger, synthetic adhesives, such as early cyanoacrylates, has been associated with issues such as toxicity, fibrous encapsulation, and chronic inflammation. There is therefore a need for surgical tissue adhesives with the bonding ability of synthetic adhesives and the bioactivity and degradation behavior of native extra-cellular matrix components.

Cyanoacrylates are a class of highly reactive 1,1-disubstituted ethylene monomers that are commonly employed as adhesives due to their fast curing rate, high bonding strength, wide range of substrates, and ease of use. Cyanoacrylate adhesives are used extensively in industrial and commercial applications and have found limited use in medicine. Monomers of cyanoacrylate are polymerizable via free radical, anionic, or zwitterionic polymerization. Anionic polymerization of the monomer can be initiated by hydroxyl ions present in moisture in the air or on the substrate surface. The early short chain alkyl cyanoacrylate adhesives were noted to have extreme inflammatory effects on tissues. Butyl cyanoacrylate, also known as Histacryl, was the first cyanoacrylate adhesive to demonstrate low tissue toxicity and good bonding strength. The toxicity of cyanoacrylates has been shown to decrease with increasing alkyl chain length. 2-Octyl cyanocrylate, commercially available as Dermabond®, has shown further improved mechanical and biological properties. Dermabond® is a registered trademark owned by Johnson & Johsnson Corp. Common medical uses of cyanoacrylates include dermal wound closure and protection. Many other applications, such as use of hemostatic agents and orthopedic adhesives, have been proposed. Advantages of cyanoacrylate adhesives over sutures or staples include lower infection rates, better cosmesis, and faster wound closure.

Current cyanoacrylate compositions are indicated only for external use. These materials result in severe histotoxicity and inflammation if embedded within the healing tissue. This is usually associated with the toxicity of the monomer, polymer, and/or degradation products, which include formaldehyde. Several compositions have been purported to improve biocompatibility and biodegradability. However, these materials may still elicit a strong, undesirable biological response and be persistent in the wound or tissue junction. Cyanoacrylate use to join living tissues can often result in obstruction of the healing path and intense foreign body response. When used for direct bonding, current cyanoacrylate adhesives bridge but also block the living tissue junction being adhered. This prevents cell migration and infiltration and replacement of the material by native tissue. A reactive, biocompatible, and biodegradable adhesive material is desired that will stabilize the tissue junction while promoting tissue infiltration, replacement, and healing through the material.

The extra-cellular matrix (ECM) that predominantly composes most tissues provides mechanical support as well as cellular signals and cues related to tissue maintenance and repair. Specific peptide domains of many components of the ECM can act as ligands for cell receptors, which can promote cell adhesion, activate downstream signaling, and affect cell behavior. These ligands have been incorporated into polymer matrices and onto material surfaces in order to promote cell adhesion, migration, and differentiation, as well as control biological performance. Background signaling from non-specific protein adsorption by a material surface must be reduced for effective and specific binding and/or signaling. This has been accomplished through the incorporation of hydrophilic polymeric components, which promote resistance to protein adsorption and fouling. For example, polyethylene glycol (PEG) grafting has been used to improve the biocompatibility of materials and extend the half-life of drugs and drug-eluting cyanoacrylate nanoparticles in-vivo. Similarly, polysaccharide grafts and PEG-cyanoacrylate co-polymers have been used to slow immunological recognition and clearance of cyanoacrylate nanoparticles. Di-cyanoacrylates of PEG and reactive PEG-functional macromers have been previously disclosed; however, the PEG domains of the polymers or co-polymers formed from these materials are more restricted than graft- or brush-like pendant chains. PEG has also been used in blends with cyanoacrylate to modify polymerization characteristics, mechanical properties, biocompatibility, and biodegradability. In these materials, PEG is not controllably grafted to the base cyanoacrylate monomer or polymer. There is a need for polymerizable adhesive macromers with non-fouling and/or cell adhesion or signaling functionality for use in tissue adhesives with improved biological performance. Additionally, an adhesive is desired that degrades in-vivo by mechanisms that do not result in the accumulation of toxic products such as formaldehyde.

ECM components are not only produced but also degraded by cells during tissue remodeling or healing. Various types of proteases, such as matrix-metalloproteases (MMP) or plasmin, cleave ECM components in a specific manner, resulting in cell-mediated degradation of the ECM. Enzyme-cleavable domains have been incorporated into synthetic polymer hydrogels to impart specific degradability by certain MMPs and allow for cell migration, infiltration and replacement of the hydrogel by tissue. Non-specifically degradable co-polymers of alpha esters have previously been incorporated into cyanoacrylate adhesives. Also disclosed are solutions of such co-polymers in monomer or co-monomers. There is still a need, however, for cyanoacrylate adhesive compositions incorporating reactive macromers or cross-linkers with enzyme-cleavable domains in order to match degradation to tissue ingrowth and healing rate.

Industrial adhesive compositions are often modified with particulate or fibrous filler material in addition to other additives in order to improve mechanical performance. Polymeric biomaterials have previously been presented that are reinforced with degradable polymer and mineral particulate and fibrous material. One example is a degradable, osteoinductive composite of mineral and biodegradable polymer. Such composites have the potential to develop morphology during degradation of the filler component. Such micro-structural morphology has been shown to strongly influence cell behavior and tissue formation in tissue engineering scaffolds.

### SUMMARY OF THE INVENTION

The present invention discloses 1,1-disubstituted electron-deficient olefin macromers, related adhesive compositions and a process for their use. Such compositions can react with moisture on surfaces or in the presence of biological surfaces or fluids, such as blood, to form a solid polymer. These adhesives can be used to create an adhesive bond between two dissimilar or similar surfaces, such as at the junction of living tissue. The inventive compositions have improved biocompatibility as well as controlled biodegradation and bioactivity. Compositions of the invention can be used as surgical adhesives to provide mechanical fixation while promoting healing across the tissue junction. The co-monomer compositions contain at least one macromer with a pendant oligomeric or linear or branched polymeric chain, which may be ester-linked to an acrylate group of the reactive olefin. The pendant polymer may be one that prevents non-specific protein adsorption, such as a polyethylene glycol (PEG). Such polymeric grafts serve to improve the biocompatibility of the resulting adhesive and decrease the toxicity of polymer degradation products. The composition may contain as co-monomers other 1,1-disubstituted electron-deficient olefins and cross-linkers such as alkyl, alkoxyalkyl, or carboxyalkyl cyanoacrylate and PEG-dicyanoacrylate, respectively. A key characteristic of the macromers and co-monomer compositions of the invention is the comb- or brush-like structure of the polymers that they form.

The composition may further contain cyanoacrylate-functionalized peptide co-macromers and cross-linkers. These may include cyanoacrylate-pendant cell binding or signaling peptide domains and cyanoacrylate-capped enzyme-cleavable domains. Another characteristic of the embodied adhesive compositions is that the polymers formed therefrom have engineered bioactivity and degradation characteristics. Non-degradable and/or non-specifically degradable cross-linking co-monomers or macromers may also be incorporated in the composition. Optionally, other co-monomers, macromers, and reactive or dead synthetic, natural, or modified natural polymers may also be included in the inventive composition, including co-polymers of the inventive macromers.

Drugs, such as antibiotic or anti-inflammatory agents, and/or biologics, such as growth hormones or gene-therapy vectors, may be incorporated in the adhesive polymer or included in the compositions containing the macromers of the present invention. These compounds may optionally be tethered to a macromer or interpenetrating polymer via a cleavable or non-cleavable linkage, or alternatively be incorporated as micro- or nano-sized particles or capsules. Polymerization and/or polymer modifying additives such as free-radical stabilizers, anionic stabilizers, initiators, accelerators, inhibitors, plasticizers, and rheology modifiers may be optionally incorporated in the composition. Formaldehyde scavenging compounds may also be included in the composition.

Furthermore, the composition may include fiber or particulate filler material to provide a reinforced polymer composite that develops a porous micro-structure upon degradation. The filler material may be composed of degradable synthetic or natural polymer, protein, mineral, or bio-glass. A releasable drug or biologic may also be included in the degradable filler material. Also disclosed are polymer/mineral cement composites including the adhesive composition, which cure by simultaneous polymerization and precipitation of mineral to form an interpenetrating composite microstructure. Micro-structural evolution, together with the cell-mediated degradation and bioactive properties of the polymer, promotes cell and tissue in-growth and allows for mechanical stabilization during healing and replacement of the material by native tissue. Also disclosed are methods of producing the monomers and adhesive compositions of the invention as well as methods of use as surgical adhesives.

### DETAILED DESCRIPTION OF THE INVENTION

The present invention is directed to adhesive compositions and methods of using the compositions. The compositions of the present invention include a 1,1-disubstituted electron-deficient olefin macromer. Monomer and co-polymer design are used to improve the degradation behavior and biological performance of cyanoacrylates. The macromers of the invention can be stabilized and stored as mono-functional precursors with well-defined properties. Additional additives can be included.

Without wanting to be limited to any one theory, it is believed that the compositions of the present invention control in-vivo degradation behavior, wound healing, and tissue regeneration. It is further believed that brush-like pendant hydrophilic functionality masks the functionalized molecule or polymer from immunological recognition and toxic degradation. It is also believed that the inclusion of minimal oligo-peptide sequences can impart specific bioactivity and cell-mediated degradation to the materials.

All patents and articles, referred to herein, are incorporated herein by reference in their entirety.

A. Macromers

The general structure for the electron deficient 1,1-disubstituted olefins is: 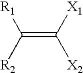
wherein R₁ and R₂ can be any group, such as H, straight or branched alkyls, but are preferably H; and
wherein X₁ and X₂ are electron-withdrawing groups such as: 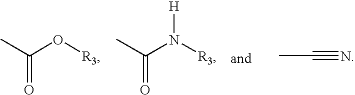
It is preferred to have at least one or both of the X₁ and X₂ to be one of the acrylates listed above.

The unique aspect of the monomers of the invention is that R₃ is a linear or branched hydrophilic or amphiphilic oligomer, polymer, or peptide with no explicit reactive functionality.

In some preferred embodiments, R₃ is methoxy poly(ethylene glycol), for example: 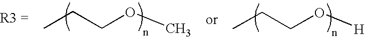
Wherein n>1, preferably n is between 2-10,000.

The resulting the monomer structure may be:

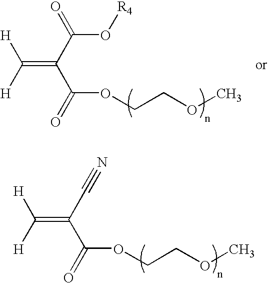

In another embodiment R₃ can be an alklyl methoxy poly(ethylene glycol): 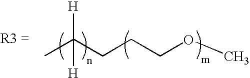
wherein n≧1, preferably n is between 1-50, and more preferably n is between 1-15; and m>1, preferably m is between 2-10,000.

The resulting monomer structure can be:

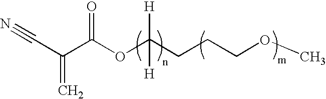

In other preferred embodiments, R₃ may be an oligo- or poly-saccharide or oligo- or polypeptide. The peptide sequence should have biological activity, in that it interacts with cells through cell-surface receptor interaction such as integrin-binding or growth-factor-like peptide domains.

The monomers are preferably combined into co-monomer compositions to control reactivity, degradability, mechanical properties, biocompatibility and bioactivity. Multiple monomers of the invention may be combined for improved performance, for example mPEG-alkyl-cyanoacrylate with a bioactive peptide-cyanoacrylate. Also monomers of the invention may be combined with known electron deficient 1,1-disubstituted olefins such as methylenemalonates, cyanoacrylamides, or cyanoacrylates.

It is believed that polymerization of the macromers of the present invention can be initiated by nucleophiles, such as an anion (e.g., hydroxyl ion), present in the moisture or on surfaces of biological surfaces or fluids, such as blood, to form a solid polymer. An example of a possible polymerization process is provided below. 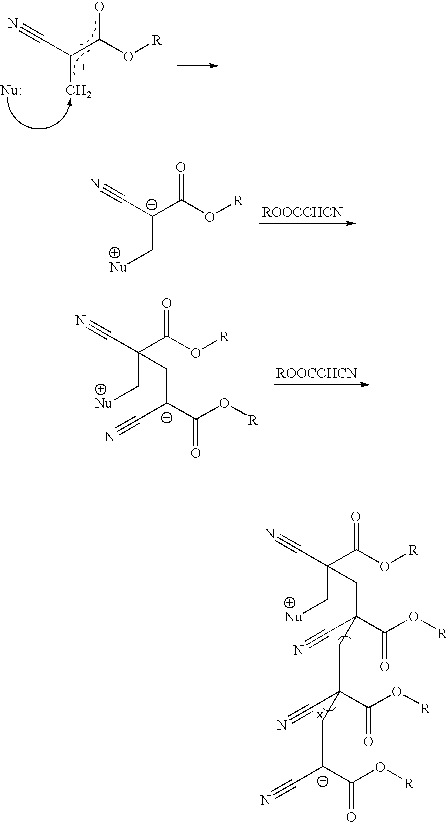
Cyanoacrylate Polymerization

Any anionic or free-radical initiator may be used to induce polymerization or polymerization may be initiated by chemical moieties at the surface of the tissue substrate or in biological fluids such as blood. Unique initiators of the invention include macromolecules with one or more functional groups specifically incorporated to induce anionic or free-radical polymerization. The distinguishing factor of these initiators is that the initiator functionality is pendant from a bio-hybrid molecule composed of synthetic and biological components such as polypeptides or polysaccharides. These initiators produce bio-hybrid co-polymers when combined with olefin functional monomers or macromers. Two specific examples include a cell-binding domain peptide sequence mono-terminated with an anionic initiating moiety and an enzyme-cleavable sequence capped at both ends with an anionic initiating moiety. Alternatively, the initiating moiety may also become activated when mixed with an activator or upon exposure to physiological environment.

Possible Synthesis Schemes of Embodied Chemical Species

Alternative PEG-Cyanoacrylate Synthesis Schemes.

1.) Transesterification of Cyanoacetic Acid with mPEG to Form mPEG-Cyanoacrylate Followed by Condensation with Formaldehyde and Thermal Cracking to mPEG Cyanoacrylate Monomer: 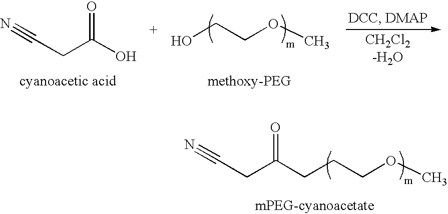 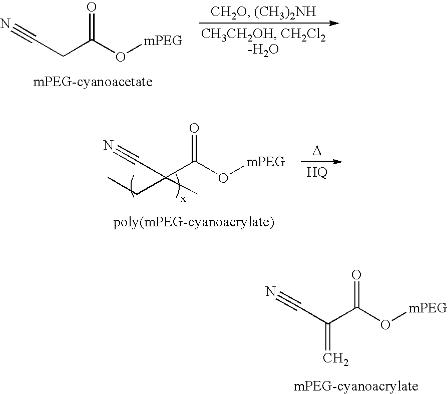
2.) Transesterification of Ethyl Cyanoacrylate to mPEG Cyanoacrylate Under Anionic Polymerization Stabilizing Acidic Conditions: 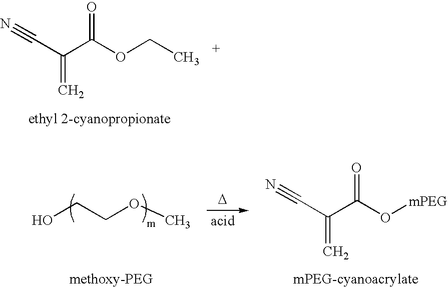
3.) Reaction of mPEG Cyanoacetic Acid with Acetylene in Presence of Catalyst to Form mPEG Cyanoacrylate: 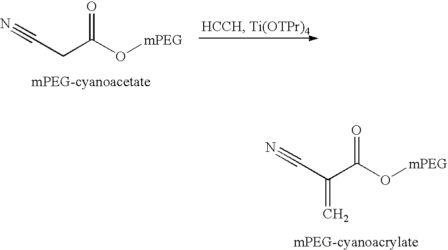
4.) Transesterification of Ethyl Cyanopropionate to Form mPEG Cyanopropionate Followed by Phenyl Selenide Substitution at Alpha Carbon and Oxidation to mPEG Cyanoacrylate: 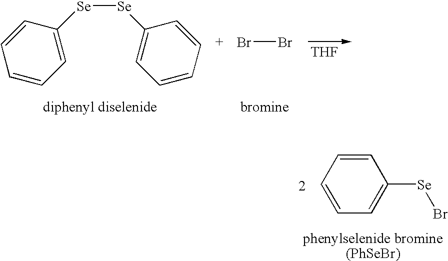 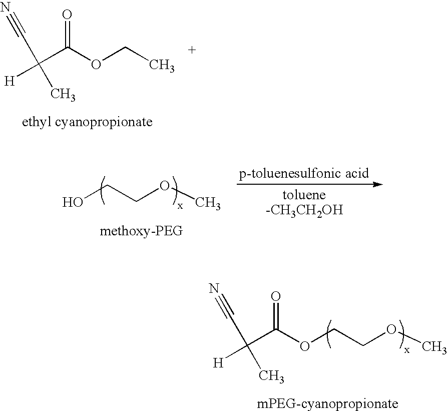 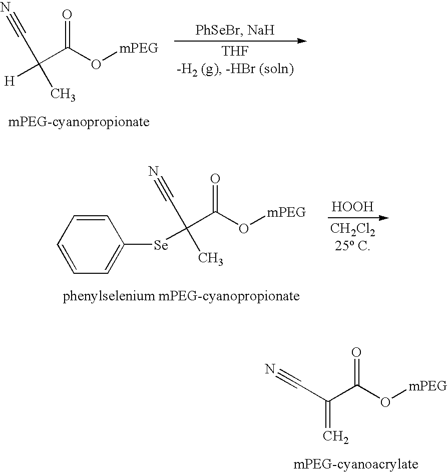
RGD-Cyanoacrylate Synthesis Scheme: 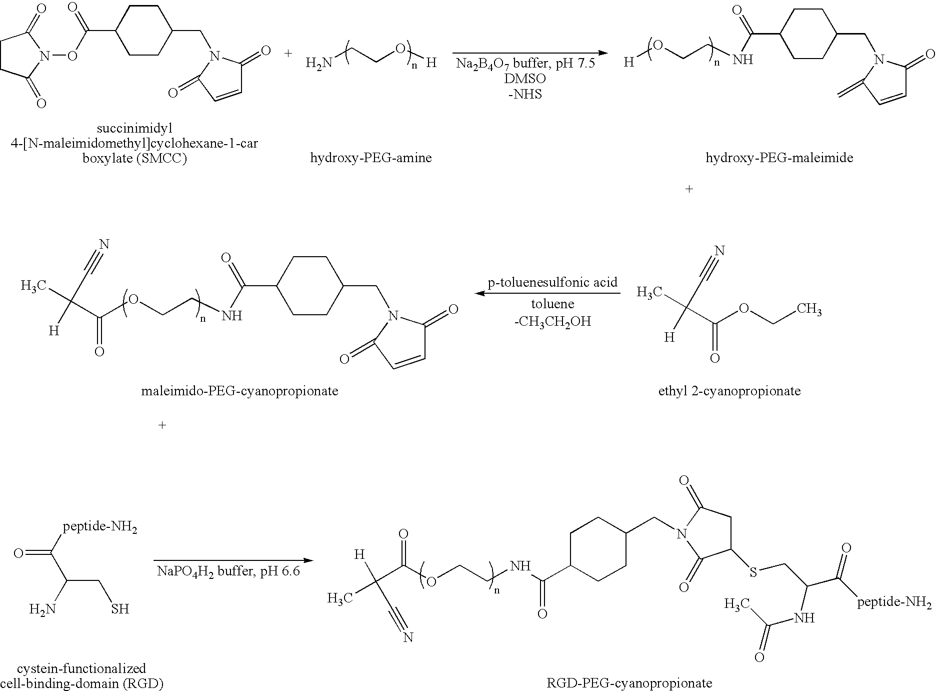 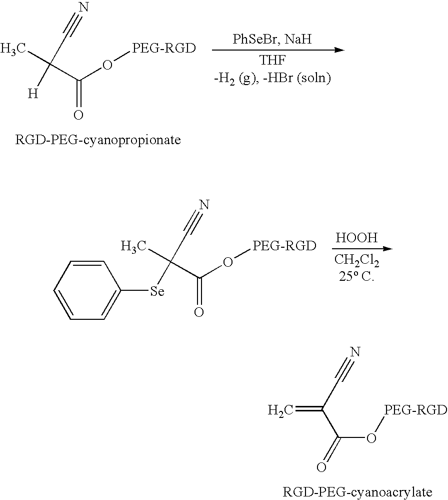
Alternative Enzyme-Cleavable Cyanoacrylate Cross-Linker Synthesis:
1.) 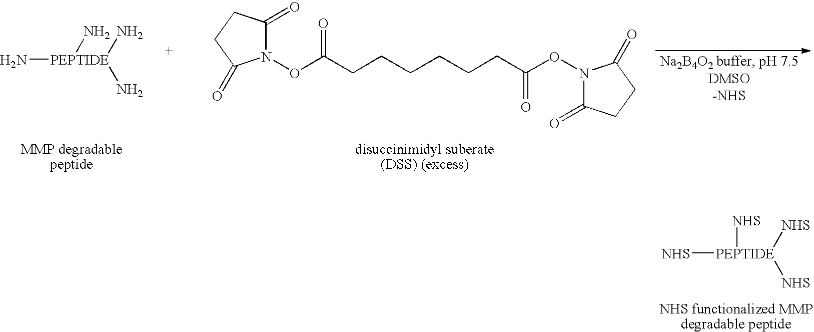 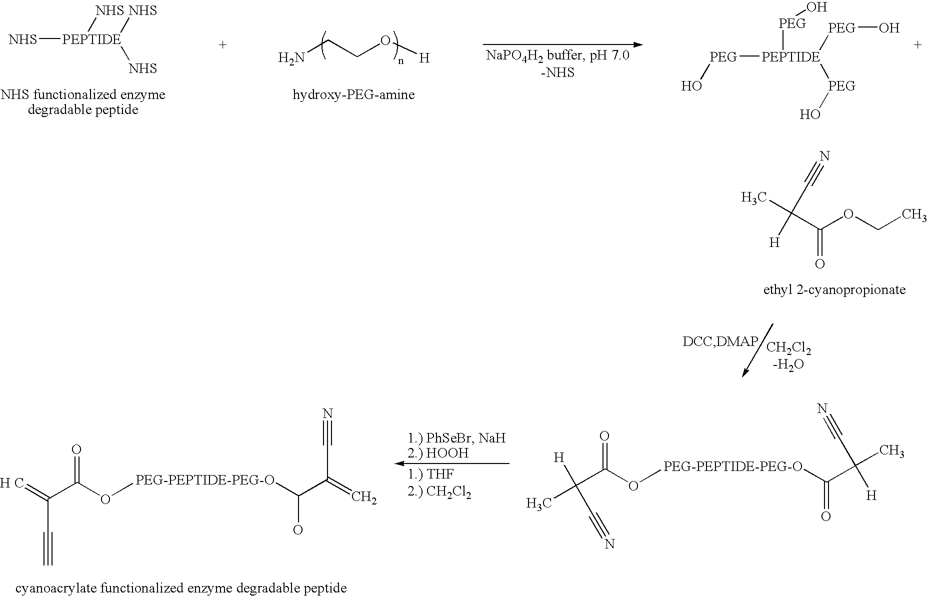
2.) 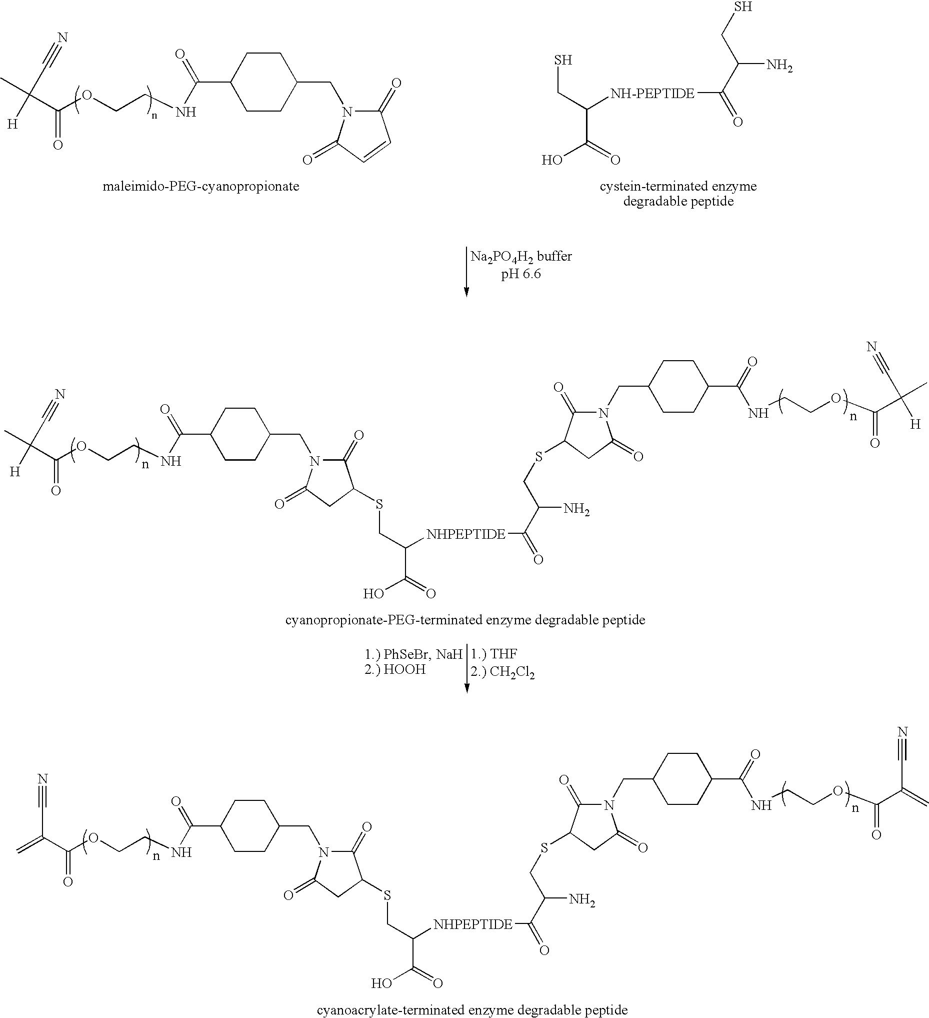
Cross-Linkers/Biological Characteristics

Cross-linkers of the invention include molecules with more than one electron-deficient olefin functionality that are specifically degradable by biological enzymes, which does not include non-specifically degradable esters. For example, protease or matrix-metalloprotease (MMP) cleavable polypeptides or minimal peptide sequences.

Previous work on materials for tissue regeneration and integration has demonstrated the powerful approach of incorporating oligopeptide domains of the extra-cellular matrix (ECM) for controlling cell behavior and material degradation. The low non-specific protein adsorption of the proposed adhesive provides a background upon which specific ligands can be presented. Peptide sequences of co-macromers can be selected for tissue specific bioactivity to promote cell and tissue infiltration and healing. These domains are intended to be presented at the surface of the material throughout degradation in order to engage cell-surface receptors and promote tissue infiltration and healing.

Other domains of ECM proteins are proteolytically degradable by specific enzymes, such matrix-metalloproteases (MMPs), which increase in production during wound healing and tissue remodeling. MMP-cleavable cross-linkers will be synthesized and incorporated in co-monomer compositions to allow for cell-mediated degradation of the adhesive. In future experimental or clinical use, this will help to ensure that strength loss due to degradation occurs at the same rate as tissue in-growth and reinforcement of the adhesive. Cell-binding and enzyme-cleavable domains for this study are chosen for their relevance to skeletal fracture healing, but could be selected for design of another tissue-specific adhesive.

Adhesives of the invention may also be modified with collagen, or other extra-cellular matrix proteins, and/or hydroxyapatite, or other calcium phosphate minerals, to improve their mechanical and biological performance.

B.) Nanoparticles

In another inventive composition, drug-loaded micro- or nano-spheres or capsules are incorporated with reactive electron-deficient olefins such as cyanoacrylates. The drug or biologic is incorporated in the particle prior to inclusion in the adhesive. This protects the drug or biologic from the reactive chemical mechanisms of the adhesive. The nanoparticles are preferably polymer or mineral nanoparticles whose synthesis and use in drug delivery are known in the art. Preferred for incorporation in the nanoparticles without limiting the invention are antibiotics, anti-inflammatories, anti-oxidant, formaldehyde-scavenging, and growth-factor components, also known in the art.

Drugs or biologics, such as proteins, can be incorporated in nano-spheres or nano-capsules to decrease drug toxicity and enzymatic degradation and allow targeted delivery. These particles exhibit drug release by surface erosion of the particle through polymer degradation. Recent advances in this field illustrate the use of co-polymer design to improve the degradation behavior and biological performance of cyanoacrylates.

C. Temperature Responsive Adhesives

Temperature responsive adhesives may be formed by including temperature responsive polymers with the reactive compositions. Temperature responsive polymers are characterized as having a lower critical solution temperature, upper critical solution temperature, melting temperature, or glass transition temperature. Preferably the transition temperature is between room temperature (20° C.) and human physiological temperature (37° C.). Polymers with a lower critical solution temperature may be incorporated with the adhesive so that the mixture gels at the tissue surface, providing improved gap-filling qualities and greater control of the liquid during application. Polymeric particles may also be included that respond to increased temperature by releasing entrapped initiators. A temperature responsive film containing reactive adhesive components can be formulated with polymers that have T_{g} or Tₘ between ambient and physiological temperature. These materials “melt” on contact with tissue, stimulating polymerization. Also, chemicals or polymers that have a strong change in pH or ionize with increased temperature may be incorporated in the composition to impart temperature-induced initiation and polymerization. The temperature responsive polymers described are known to the art (Galaev I Y, Mattiasson B. ‘Smart’ polymers and what they could do in biotechnology and medicine. Trends Biotechnol. 1999 August; 17(8):335-40, which is incorporated herein by reference) and molecular and polymer engineering can be used to modify their behavior (Jeong B, Outowska A. Lessons from nature: stimuli-responsive polymers and their biomedical applications. Trends Biotechnol. 2002 July; 20(7):305-11, which is incorporated herein by reference).

D. Semi-Interpenetrating Polymer Networks (SIPNS) or Interpenetrating Polymer Networks (IPNS)

IPNs or sIPNs may be formed by including peptide functionalized and/or enzyme-cleavable polymers or networks in with the reactive compositions. The bio-hybrid polymers may alternatively be mixed with the adhesive prior to application.

E. Additives

Other additives to the compositions are known in the art and may be found in U.S. Pat. No. 6,174,919, which is incorporated herein by reference in its entirety.

F. Cement Composites

Polymers capable of chelating or being cross-linked by divalent cations may be incorporated in calcium phosphate and related cements in order to improve adhesion and mechanical performance such as toughness. Examples of such polymers include but are not limited to poly(vinyl pyrrolidone), poly(vinyl caprolactam), poly(anhydrides) or related polyacids such as poly(maleic anhydride) and poly(fumarates), poly(itaconic acid), substituted poly(phosphazines), and poly(methylene malonic acid). Co-polymers of these polymers with oligopeptides or other bioactive component functionality may provide further improve biological performance. These cements usually set by an acid-base reaction between two solutions that are mixed and pH increases during cement reaction and hardening. pH- or temperature-sensitive polymer spheres or capsules containing the strength-enhancing polymer may be included in one of the solutions that release their components during cement setting. Such cements and polymer-cement composites may additionally be formulated with reactive monomers or macromers. pH increase may induce anionic initiation and polymerization or heat release may induce free-radical polymerization. Responsive polymers containing initiators may also be included in the composition.

What is claimed is:

1. A composition for use as an adhesive for tissue comprising,
a monomer having the general formula 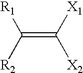
wherein R₁ and R₂ are independently selected from the group consisting of hydrogen, alkyls, aryls, phenols, halides, oligomers, polysaccharides, peptide grafts and proteins;
wherein X₁ and X₂ are independently selected from the group consisting of 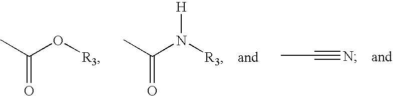
wherein R₃ is selected from the group consisting of linear and branched hydrophilic oligomer, linear and branched amphiphilic oligomer, linear and branched hydrophilic polymer, linear and branched amphiphilic polymer, linear and branched hydrophilic peptide, linear and branched amphiphilic peptide, linear and branched hydrophilic polypeptide, linear and branched amphiphilic polypeptide, linear and branched hydrophilic polysaccharides, and linear and branched amphiphilic polysaccharide.

2. The composition according to claim 1, wherein R₃ is a linear hydrophilic polymer with a general structure selected from the group consisting of: 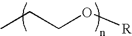
wherein n>1; and 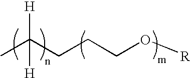
wherein n≧1, and m>1; and R=H=hydrogen, methyl group, or peptide sequence.

3. The composition according to claim 1, wherein the monomer is a cross-linker wherein R₃= 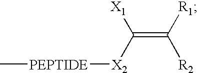
wherein the peptide sequence is selected to be enzymatically biodegradable;
wherein R₁ and R₂ are independently selected from the group consisting of hydrogen, alkyls, aryls, phenols, halides, oligomers, polysaccharides, peptide grafts and proteins; and
wherein X₁ and X₂ are independently selected from the group consisting of

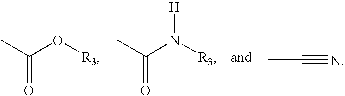

4. The composition of claim 1, further comprising reinforcing filler.

5. The composition of claim 4 wherein the reinforcing filler is in the form of particles.

6. The composition of claim 5 wherein the particles are microparticles.

7. The composition of claim 5 wherein the particles are nanoparticles.

8. The composition of claim 1, further comprising reinforcing filler in the form of fibers.

9. The composition of claim 8 wherein the fibers are microfibers.

10. The composition of claim 8 wherein the fibers are nanofibers.

11. The composition of claim 4 wherein the filler contains an additive selected from the group consisting of a drug, a biologically active compound, and a formaldehyde scavenging agent.

12. The composition of claim 6 wherein the filler is bioactive.

13. The composition of claim 1 further comprising a thermo-responsive polymer that exhibits a phase transition temperature between 0° C. and 37° C.

14. The composition of claim 13, wherein the thermo-responsive polymer releases a polymerization initiator.

15. The composition of claim 1, further comprising an admixture.

16. The composition of claim 1, further comprising an interpenetrating network with a peptide-modified bioactive.

17. The composition of claim 1, further comprising an interpenetrating network with an enzymatically degradable co-polymer.

18. The composition of claim 1, further comprising an admixture with hydraulic cement.

19. A surgical adhesive comprising a hydraulic cement capable of forming at least one divalent cation, and a polymer capable of chelating or being cross-linked when exposed to the at least one divalent cation.

20. The composition of claim 19, wherein the polymer is poly(vinyl pyrrolidone), poly(vinyl caprolactam), poly(anhydrides) or polyacids such as poly(maleic anhydride) and poly(fumarates), poly(itaconic acid), substituted poly(phosphazines), and poly(methylene malonic acid).

21. The composition of claim 19, wherein the polymer is a peptide-functional co-polymer such that the peptide binds cell-surface receptors.

22. The composition of claim 19, wherein the polymer is a peptide-functional co-polymer such that the peptide is enzymatically active.

23. The composition of claim 19, wherein the polymer is a peptide-functional co-polymer such that the peptide is cleavable.

24. The composition of claim 19, wherein the polymer is separately encapsulated from the hydraulic cement, and wherein the polymer is released during cement setting.

25. The composition of claim 19, further comprising a reactive monomer or pre-polymer.

26. The composition of claim 25, wherein a change in pH or temperature upon cement setting initiates polymerization of the reactive monomer or pre-polymer.

27. A composition for use as an adhesive for tissue comprising a monomer having the chemical structure: 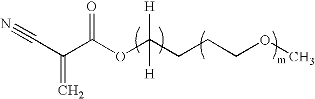
wherein m and n>=1.

28. A composition for use as an adhesive for tissue comprising a monomer having the chemical structure: 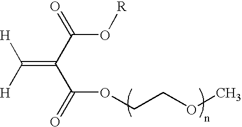
wherein R is a hydrogen or alkyl group and n>=1.

29. A composition for use as an adhesive for tissue comprising a monomer having the chemical structure: 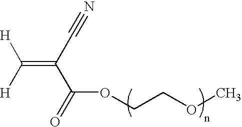
wherein n>=1.

30. A process for bonding tissue comprising the steps of:
(a) applying a surgical adhesive containing a reactive electron-deficient olefin to one tissue surface; and
(b) joining with another tissue surface.
